(19) United States
(12) Patent Application Publication
(10) Pub. No.: US 2021/0298330 A1
(43) Pub. Date: Sep. 30, 2021

(54) ORAL SOLUTION HAVING HIGH SOD ENZYMATIC ACTIVITY AND PREPARATION METHOD THEREFOR

(71) Applicant: INNER MONGOLIA GOLD LAND INDUSTRIAL CO., LTD., Inner Mongolia (CN)

(72) Inventors: Xiaojun LIAO, Beijing (CN); Zhiqiang HOU, Beijing (CN); Liang ZHAO, Beijing (CN); Yongtao WANG, Beijing (CN)

(21) Appl. No.: 17/264,841

(22) PCT Filed: Nov. 1, 2018
(86) PCT No.: PCT/CN2018/113302

§ 371 Date: Jan. 31, 2021

(30) Foreign Application Priority Data
Aug. 2, 2018 (CN) 201810870009.6

(51) Int. Cl. A23L 2/04; A23L 29/00

(52) U.S. Cl. CPC A23L 2/04; A23L 29/06

(57) ABSTRACT

The invention relates to an oral solution having high SOD enzymatic activity and the preparation method therefor, wherein the method comprises: pressing edible plant raw materials, followed by removing large particles by means of centrifugation (1000-4000 rpm, 0-30 min); subsequently, separating the supernatant using two processes of ultrafiltration (the first separation adopting a tubular membrane with pore size ranging from 60-120 kD; the second accomplished by an spiral-wound membrane with pore size ranging from 8-30 kD), before treated using ultra-high pressure at 200-600 MPa for a period of 1-20 min. After treated by the method of the invention, SOD has been activated with enhanced enzymatic activity up to above 10000 U/ml, which has addressed problems related to low enzymatic activity of SOD in products. The products obtained by the present method are more beneficial to human health.

### CROSS-REFERENCE TO RELATED APPLICATIONS

This application is a national stage application of PCT/CN2018/113302. This application claims priorities from PCT Application No. PCT/CN2018/113302, filed Jan. 11, 2018, and from the Chinese patent application 201810870009.6 filed Aug. 2, 2018, the content of which is incorporated herein in the entirety by reference.

### FIELD

The present invention belongs to the biological field, and specifically relates to an oral solution having high SOD enzymatic activity and the preparation method therefor.

### BACKGROUND

Superoxide dismutase (abbreviated as SOD) is a type of redox enzyme widely existing in a great variety of organisms. SOD is a kind of metalloenzyme that has superoxide anion free radical scavenging activity, which can efficiently protect the body from the damage caused by superoxide anion free radical, and can be used as a pharmaceutical enzyme with a wide range of therapeutic effects.

Up to now, SOD has been identified in a great variety of organisms, including bacteria, fungi, alga, insects, fishes, plants, and mammals. At present, these SODs can be divided into four types, including Cu/Zn-SOD, Mn-SOD, Fe-SOD, and Ni-SOD, based on their metal prosthetic groups. Although plant-derived SOD has been proved to possess positive effect on human health, its enzymatic activity per weight or volume of plant is relatively low. Common plant resources for SOD include sea buchthorn, kiwi fruit, mulberry, blueberry, and roxburgh rose etc. In previous studies, SOD has been isolated by DEAE ion-exchanger from plant fruits, which has molecular weights from 30 to 35 kDa. However, the existing separation process using DEAE ion-exchanger has disadvantages, such as higher cost, longer separation duration and lower yield of product, thereby making it difficult for the large-scale production.

### SUMMARY

To address the above technical problems, the present invention provides an oral solution having high SOD enzymatic activity and the preparation method therefor. After treated by the present method, the SOD enzymatic activity in products is high and stable, which has improved the health-care function of the products, and efficiently solved problems related to the loss and instability of SOD enzymatic activity during processing. Thus, the products obtained by the present method are more beneficial to human health.

The preparation method provided by the present invention for an oral solution having high SOD enzymatic activity comprises the following steps:

1) edible plant raw materials are pressed to obtain plant juice;

2) the plant juice is centrifuged, and the supernatant is collected;

3) the supernatant is separated by two processes of ultrafiltration, and the filtrate is collected;

4) the filtrate is treated by ultra-high pressure to obtain liquid with high SOD enzymatic activity; and

5) the liquid with high SOD enzymatic activity is prepared to an oral solution.

In step 1) of the method above, the edible plant raw materials can be selected from the group consisting of roots, stems, leaves and fruits of the edible plants, and specifically, can be the fruits from one or more of blueberry, kiwi fruit, mulberry, sea buchthorn and roxburgh rose etc.

The juice preparation process can specifically include crushing and pressing.

In step 2), the processing conditions for the centrifugation include: 1000-4000 rpm, and 0-30 min (both endpoints excluded); and specifically, the conditions can be 3000 rpm and 10 min.

In step 3), the two processes of ultrafiltration adopt membranes with the pore size ranging from: 60-120 kD for the tubular membrane, and 8-30 kD for the spiral-wound membrane; and specifically, the pore size for the ultrafiltration membranes can be: 100 kD for the tubular membrane, and 10 kD for the spiral-wound membrane.

In step 4), the pressure for the ultra-high pressure treatment can be 200-600 MPa, and the duration can be: 1-20 min, and specifically, the pressure for the ultra-high pressure treatment can be 550 MPa, and the duration can be 5 min.

The temperature for the ultra-high pressure treatment can be in the range from 0 to 40° C.

Preferably, the pressure medium for the ultra-high pressure treatment is water.

In step 5), the liquid with high SOD enzymatic activity can be prepared to an oral solution by conventional methods known in the art.

The oral solution prepared by the above method with high SOD enzymatic activity also falls into the protection scope of the present invention.

The content of soluble solid in the oral solution with high SOD enzymatic activity is from 2 to 15° Brix, with pH in the range from 2.0 to 6.0

The enzyme SOD can be activated by the process above, which increases the SOD enzyme activity in the oral solution, and supports the health-care function of the products.

The beneficial effects of the present invention include:

1) In the present invention, the oral solution has been separated by centrifugation together with two processes of ultrafiltration using membranes with different molecular weight cut-off values, which efficiently increases the enzymatic activity and greatly improves the health-care function of the products.

2) In the present invention, the enzyme SOD has been activated by ultra-high pressure, leading to higher enzymatic activity.

3) As compared with conventional separation method using DEAE ion-exchanger, the technique using in the present invention has the following advantages, including lower cost, higher efficiency, and excellent practicality, etc.

### The Best Mode to Implement the Invention

Unless otherwise indicated, the experiment procedures in Examples below are conventional in the art.

### EXAMPLE 1

A preparation method for an oral solution with high SOD enzymatic activity comprises crushing and pressing of blueberry, mulberry, sea buchthorn, and roxburgh rose, centrifugation, ultrafiltration, and ultra-high pressure treatment. Specifically, the method comprises the following steps: the juice prepared from the plant fruits is aliquoted into suitable containers, and centrifuged under the conditions described below; the supernatant is then separated by two processes of ultrafiltration; and the filtrate is subjected to ultra-high pressure using water as the pressure medium.

Among others, the processing method for the oral solution comprises the following steps:
(1) eFrsh or frozen fruit raw materials with no spoilage from blueberry, mulberry, sea buchthorn, and roxburgh rose are adopted;
(2) The materials are cleaned by washing;
(3) The fruits are crushed and pressed to obtain juice;
(4) The juice is obtained by filtration using 4-layer gauze to make the hide trimmings and the juice separated.
(5) The filtrate is centrifuged under the following conditions: 3000 rpm, and 10 min.
(6) The supernatant is separated by ultrafiltration using 100 kD tubular membrane and 10 kD spiral-wound membrane.
(7) The filtrate is subjected to ultra-high pressure treatment under the pressure of 550 MPa for 5 min.

The content of soluble solid in the oral solution with high SOD enzymatic activity prepared by such method is about 8° Brix with pH 3.7.

### EXAMPLE 2

A preparation method for an oral solution with high SOD enzymatic activity comprises crushing and pressing of blueberry, kiwi fruit, sea buchthorn, and roxburgh rose, centrifugation, ultrafiltration, and ultra-high pressure treatment. Specifically, the method comprises the following steps: the juice prepared from raw materials is aliquoted into suitable containers, and centrifuged under the conditions described below; the supernatant is then separated by ultrafiltration; and the filtrate is subjected to ultra-high pressure treatment.

Among others, the processing method for the oral solution comprises the following steps:
(1) Fresh or frozen fruit raw materials with no spoilage from blueberry, kiwi fruit, sea buchthorn, and roxburgh rose are adopted;
(2) The materials are cleaned by washing;
(3) The fruits are transferred to a screw extractor to prepare juice;
(4) The raw juice is obtained by filtration using 4-layer gauze to make kernels, hide trimmings and the juice separated;
(5) The filtrate is centrifuged under the following conditions: 3000 rpm, and 10 min.
(6) The supernatant is separated by ultrafiltration using 100 kD tubular membrane and 10 kD spiral-wound membrane.
(7) The filtrate is subjected to ultra-high pressure treatment under the pressure of 550 MPa for 5 min.

The content of soluble solid in the oral solution with high SOD enzymatic activity prepared by such method is about 10° Brix with pH 2.7.

### COMPARATIVE EXAMPLE 1-1

A processing method for fruit juice is employed, which is different from the method of Example 1 only by that the following procedures are not used, including concentration by ultrafiltration and ultra-high pressure treatment.

### COMPARATIVE EXAMPLE 1-2

A processing method for fruit juice is employed, which is different from the method of Example 1 by that only concentration by ultrafiltration is used, and the ultra-high pressure treatment is not.

### COMPARATIVE EXAMPLE 1-3

A processing method for fruit juice is employed, which is the same as the method of Example 1 except that no concentration by ultrafiltration is used.

### COMPARATIVE EXAMPLE 1-4

A processing method for fruit juice is employed, which is the same as the method of Example 1 except for the separation conditions by ultrafiltration, i.e., only 100 kD tubular membrane is used.

### COMPARATIVE EXAMPLE 2-1

A processing method for fruit juice is employed, which is different from the method of Example 1 only by that the following procedures are not used, including concentration by ultrafiltration and ultra-high pressure treatment.

### COMPARATIVE EXAMPLE 2-2

A processing method for fruit juice is employed, which is different from the method of Example 1 by that only concentration by ultrafiltration is used, and the ultra-high pressure treatment is not.

### Conclusion 1

The enzymatic activities of SOD are determined for the fruit and vegetable juice prepared in Example 1 and Comparative example 1, respectively. The results are listed in Table 1 below. The activity of SOD is determined using WSTT assay (see, e.g., Peskin, A. V., & Winterbourn, C. C. (2000). A microtiter plate assay for superoxide dismutase using a water-soluble tetrazolium salt (WST-1). Clinica Chimica Acta, 293(1-2), 157-166). WST-1 is short for 2-(4-Iodophenyl)-3-(4-nitrophenyl)-5-(2,4-disulfophenyl)-2H-5 tetrazolium, monosodium salt. WST-1 can react with the superoxide anion generated by the reaction catalyzed via xanthine oxidase to give water soluble formazan dye, which can be suppressed by SOD. The enzymatic activity of SOD can be determined through colorimetric analysis for the product of WST-1.

TABLE 1

| Item | SOD enzymatic activity U/mL |
|---|---|
| Example 1 | 11052.7 |
| Comparative example 1-1 | 6102.5 |
| Comparative example 1-2 | 9257.4 |
| Comparative example 1-3 | 6832.5 |
| Comparative example 1-4 | 6958.5 |

As indicated by the results listed in Table 1, after concentration by ultrafiltration, the SOD enzymatic activity is enhanced by about 34% (9257.4 U/mL) as compared with Comparative example 1-1; after ultrafiltration, the SOD can be further activated by ultra-high pressure treatment, making the enzymatic activity enhanced again by about 20%, exceeding 10000 U/mL. The results have indicated that the preparation method of the invention has significantly enhanced SOD enzymatic activity in the fruit and vegetable juice.

### Conclusion 2

The enzymatic activities of SOD are determined for the fruit juice prepared in Example 2, Comparative example 2-1, and Comparative example 2-2, respectively. The activity of SOD is determined by the same method as Example 1. The results were listed in Table 2 below.

TABLE 2

| | SOD enzymatic activity U/mL |
|---|---|
| Example 2 | 10091.13 |
| Comparative example 2-1 | 5892.7 |
| Comparative example 2-2 | 8072.9 |

As indicated by the results listed in Table 2, after concentration by ultrafiltration, the SOD enzymatic activity in the fruit juice is enhanced by about 37% (8072.9 U/mL) as compared with Comparative example 2-1; the SOD can be further activated by concentration using ultrafiltration+ultra-high pressure treatment, making the enzymatic activity enhanced again by about 25% as compared with Comparative example 2-2, reaching 10091.13 U/mL. The results have indicated that the preparation method of the invention has significantly enhanced SOD enzymatic activity in the fruit and vegetable juice.

### INDUSTRIAL APPLICATIONS

The enzyme SOD can be activated by the process of the invention, which increases the SOD enzyme activity in the oral solution, and supports the health-care function of the products.

In the present invention, the oral solution has been separated using centrifugation together with two processes of ultrafiltration using membranes with different molecular weight cut-off values, which efficiently increases the enzymatic activity and greatly improves the health-care function of the products. In the present invention, the enzyme SOD has been activated by ultra-high pressure, leading to higher enzymatic activity; as compared with conventional separation method using DEAE ion-exchanger, the technique using in the present invention has the following advantages, including lower cost, higher efficiency, and excellent practicality, etc.

1. A preparation method for an oral solution having high SOD enzymatic activity, comprising the following steps:
1) edible plant raw materials are pressed to obtain plant juice;
2) the plant juice is centrifuged, and the supernatant is collected;
3) the supernatant is separated by two processes of ultrafiltration, and the filtrate is collected;
4) the filtrate is treated by ultra-high pressure to obtain liquid with high SOD enzymatic activity; and
5) the liquid with high SOD enzymatic activity is prepared to an oral solution.

2. The method according to claim 1, wherein in step 1), the edible plant raw materials are selected from the group consisting of roots, stems, leaves and fruits of the edible plants; and the juice preparation process includes crushing and pressing.

3. The method according to claim 1, wherein in step 2), the conditions for the centrifugation include: 1000-4000 rpm, and 0-30 min.

4. The method according to claim 1, wherein in step 3), the two processes of ultrafiltration adopt membranes with the pore size ranging from: 60-120 kD for the tubular membrane, and 8-30 kD for the spiral-wound membrane.

5. The method according to claim 1, wherein in step 4), the pressure for the ultra-high pressure treatment is from 200 to 600 MPa, with a duration in the range of: 1-20 min.

6. The method according to claim 1, wherein in step 4), the temperature for the ultra-high pressure treatment is in the range of 0-40° C.; the pressure medium for the ultra-high pressure treatment is water.

7. The oral solution with high SOD enzymatic activity prepared by the method according to claim 1, wherein the content of soluble solid in the oral solution ranges from 2 to 15° Brix, with pH from 2.0 to 6.0.

8. The method according to claim 2, wherein in step 2), the conditions for the centrifugation include: 1000-4000 rpm, and 0-30 min.

9. The method according to claim 2, wherein in step 3), the two processes of ultrafiltration adopt membranes with the pore size ranging from: 60-120 kD for the tubular membrane, and 8-30 kD for the spiral-wound membrane.

10. The method according to claim 3, wherein: in step 3), the two processes of ultrafiltration adopt membranes with the pore size ranging from: 60-120 kD for the tubular membrane, and 8-30 kD for the spiral-wound membrane.

11. The method according to claim 2, wherein: in step 4), the pressure for the ultra-high pressure treatment is from 200 to 600 MPa, with a duration in the range of: 1-20 min.

12. The method according to claim 3, wherein: in step 4), the pressure for the ultra-high pressure treatment is from 200 to 600 MPa, with a duration in the range of: 1-20 min.

13. The method according to claim 4, wherein in step 4), the pressure for the ultra-high pressure treatment is from 200 to 600 MPa, with a duration in the range of: 1-20 min.

14. The method according to claim 2, wherein in step 4), the temperature for the ultra-high pressure treatment is in the range of 0-40° C.; the pressure medium for the ultra-high pressure treatment is water.

15. The method according to claim 3, wherein in step 4), the temperature for the ultra-high pressure treatment is in the range of 0-40° C.; the pressure medium for the ultra-high pressure treatment is water.

16. The method according to claim 4, wherein in step 4), the temperature for the ultra-high pressure treatment is in the range of 0-40° C.; the pressure medium for the ultra-high pressure treatment is water.

17. The method according to claim 5, wherein in step 4), the temperature for the ultra-high pressure treatment is in the range of 0-40° C.; the pressure medium for the ultra-high pressure treatment is water.

18. The oral solution with high SOD enzymatic activity prepared by the method according to claim 2, wherein the content of soluble solid in the oral solution ranges from 2 to 15° Brix, with pH from 2.0 to 6.0.

19. The oral solution with high SOD enzymatic activity prepared by the method according to claim 3, wherein the content of soluble solid in the oral solution ranges from 2 to 15° Brix, with pH from 2.0 to 6.0.

20. The oral solution with high SOD enzymatic activity prepared by the method according to claim 4, wherein the content of soluble solid in the oral solution ranges from 2 to 15° Brix, with pH from 2.0 to 6.0.
